(19) United States
(12) Patent Application Publication
(10) Pub. No.: US 2018/0268101 A1
(43) Pub. Date: Sep. 20, 2018

(54) LINKAGE DISEQUILIBRIUM METHOD AND DATABASE

(71) Applicant: Sirona Genomics, Inc., Mountain View, CA (US)

(72) Inventors: Chunlin WANG, Mountain View, CA (US); Ming LI, Mountain View, CA (US)

(73) Assignee: Sirona Genomics, Inc., Mountain View, CA (US)

(21) Appl. No.: 15/764,107

(22) PCT Filed: Sep. 28, 2016
(86) PCT No.: PCT/US16/54166

§ 371 Date: Mar. 28, 2018

Related U.S. Application Data
(60) Provisional application No. 62/233,712, filed on Sep. 28, 2015.
(60) Provisional application No. 62/284,356, filed on Sep. 28, 2015.
(60) Provisional application No. 62/399,707, filed on Sep. 26, 2016.

(51) Int. Cl. G06F 19/18; C12Q 1/6881; C12Q 1/6844; G06F 19/28; C12Q 1/6883; G06F 19/22

(52) U.S. Cl. CPC G06F 19/18; C12Q 1/6881; G06F 19/22; G06F 19/28; C12Q 1/6883; C12Q 1/6846

(57) ABSTRACT

A method for determining the association of Human Leukocyte Antigen (HLA) alleles at adjacent loci in genomic DNA from a biological sample obtained from a human subject is disclosed. A method and system of validating correctness of an assignment of an allele variant to a genetic human leucocyte antigen (HLA) locus are also disclosed.

### RELATED APPLICATIONS

This application claims priority of U.S. Provisional Application No. 62/233,712, filed on Sep. 28, 2015, U.S. Provisional Application No. 62/284,356, filed on Sep. 28, 2015, and U.S. Provisional Application No. 62/399,707, filed on Sep. 26, 2016. The entirety of the provisional applications is incorporated herein by reference.

### FIELD

The present disclosure generally relates to determining the haplotype of an individual from genomic sequence information.

### BACKGROUND

In population genetics, linkage disequilibrium (LD) is the non-random association of alleles at different loci, i.e., the presence of statistical associations between alleles at different loci that are different from what would be expected if alleles were independently, randomly sampled based on their individual allele frequencies. Human Leucocyte Antigen (HLA) constitutes a group of cell surface antigens as the Major Histocompatibility Complex (MHC) of humans. Because HLA genes are located at adjacent loci on the particular region of a chromosome and presumed to exhibit epistasis with each other or with other genes, a sizable fraction of alleles are in linkage disequilibrium. Therefore, HLA LD information is useful for evaluating genotype results.

HLA typing results are critically important to a number of clinical applications including transplantation. Individuals undergoing transplantation procedures, the mistyping of an individual's HLA profile could lead to serious deleterious effects. Accordingly, there is a great need for processing methods that identify and assign alleles from sequence data and incorporate allele associations where the processing methods exhibit high fidelity.

### SUMMARY

A first aspect of the present disclosure relates to a method for determining the association of Human Leukocyte Antigen (HLA) alleles at adjacent loci in genomic DNA from a biological sample obtained from a human subject. The method comprises the steps of amplifying a segment of genomic DNA from the sample by long-range PCR reaction; sequencing the amplified DNA; determining the association frequency of the genotype of an allele of a first locus with the genotype of an allele of at least one adjacent locus by reference to a database of loci associations; and reporting an association score of said allele in a first locus with the genotype of the allele in at least one adjacent locus, thereby determining the association of Human Leukocyte Antigen (HLA) alleles at adjacent loci in genomic DNA.

A second aspect of the present disclosure relates to a database matrix for use in the analysis of association score of an allele to be assigned to a first locus and at least one additional locus. The database matrix comprises a field corresponding to an allele to be genotyped; at least one other field corresponding to another allele at a different locus; at least another field corresponding to the probability of the allele to be genotyped to a locus and the at least one other field as expressed as a probability.

A third aspect of the present disclosure relates to a method of validating correctness of an assignment of an allele variant to a genetic human leucocyte antigen (HLA) locus. The method comprises acquiring genotype information representing an assignment of an allele variant to a genetic human leucocyte antigen (HLA) locus; determining a score representing an association of the allele variant with at least one other allele variant of at least one adjacent genetic locus; using the determined score to generate an indication indicating correctness of the assignment of the allele variant to the genetic HLA locus; and reporting the indication.

A fourth aspect of the present disclosure relates to a method of operating a computing device comprising at least one processor, the method comprising: executing the at least one processor to acquire genotype information representing an assignment of an allele variant to a genetic human leukocyte antigen (HLA) locus; and when it is determined that a score representing an association of the allele variant with at least one other allele variant of at least one adjacent genetic locus exists using the determined score to generate an indication indicating the likelihood of the correctness of the assignment of the allele variant to the genetic HLA locus; and reporting the indication.

A fifth aspect of the present disclosure relates to a computing system comprising at least one processor configured to assign at least one partial haplotype to a genetic locus by performing the method according to any one of the first through fourth aspects and their embodiments.

A sixth aspect of the present disclosure relates to a system for validating correctness of a genotype of a sample obtained from a subject, the system comprising: at least one processor; a memory communicatively coupled to the processor, the memory having stored thereon computer executable instructions that, when executed by the at least processor, perform a method comprising: acquiring genotype information representing an assignment of an allele variant to a genetic human leukocyte antigen (HLA) locus; determining a score representing an association of the allele variant with at least one other allele variant of at least one adjacent genetic locus; using the determined score to generate an indication indicating correctness of the assignment of the allele variant to the genetic HLA locus; and reporting the indication.

A seventh aspect of the present disclosure relates to at least one non-transitory computer storage device storing computer-executable instructions that, when executed by at least one processor, cause the at least one processor to perform a method comprising: acquiring a genotype information representing an assignment of an allele variant to a genetic human leucocyte antigen (HLA) locus; acquiring a score representing an association of the allele variant with at least one other allele variant of at least one adjacent genetic locus; using the acquired score to generate an indication indicating correctness of the assignment of the allele variant to the genetic HLA locus; and reporting the indication.

An eighth aspect of the present disclosure relates to a method of operating a computing device comprising at least one processor, the method comprising: executing the at least one processor to acquire genotype information representing an assignment of an allele variant to a genetic human leucocyte antigen (HLA) locus; accessing a computer readable storage device storing linkage disequilibrium information to determine whether the linkage disequilibrium information includes a score representing an association of the allele variant with at least one other allele variant of at least one adjacent genetic locus; when it is determined that the linkage disequilibrium information includes the score using the determined score to generate an indication indicating correctness of the assignment of the allele variant to the genetic HLA locus; and reporting the indication.

A ninth aspect of the present disclosure relates to a method of assigning an allele to a genetic locus comprising: amplifying coding and non-coding DNA from a genetic locus from a sample of genomic DNA to produce an amplicon; sequencing the amplicon; identifying at least a first allele variant and a second allele variant of the genetic locus from the amplicon; determining a score representing an association of the first allele variant with at least one other allele variant of at least one adjacent genetic locus; and using the determined score to generate an indication indicating correctness of the assignment of the allele variant to the genetic HLA locus.

### BRIEF DESCRIPTION OF THE DRAWINGS

The present invention can be better understood by reference to the following drawings. The drawings are merely exemplary to illustrate certain features that may be used singularly or in combination with other features and the present invention should not be limited to the embodiments shown.

FIG. 1 shows an exemplary scheme for long-range PCR amplification of HLA genes from human genomic DNA.

### DETAILED DESCRIPTION OF THE INVENTION

The following detailed description is presented to enable any person skilled in the art to make and use the invention. For purposes of explanation, specific nomenclature is set forth to provide a thorough understanding of the present invention. However, it will be apparent to one skilled in the art that these specific details are not required to practice the invention. Descriptions of specific applications are provided only as representative examples. The present invention is not intended to be limited to the embodiments shown, but is to be accorded the widest possible scope consistent with the principles and features disclosed herein.

The present disclosure relates to an improved method of allele assignment to a genetic locus by determining an association score of the allele of interest with other alleles. It has been determined that an allele is present more frequently with some alleles than with others in what is termed in the art a “linkage disequilibrium” Here, the inventors developed an improved method of allele assignment using a method of calculating an association score of the allele of interest together with at least one other allele, adjacent or near the allele of interest to determine if the allele of interest is highly likely to be present with other alleles in other loci. This association score, often represented by the −log probability of the association is evaluated to determine if the allele of interest and the associated allele(s) are in linkage disequilibrium. If so, then the method of assigning an allele to a locus is improved by increasing the confidence of the allele assignment based on another metric.

Information for the LD database forming the association table (or LD table) is collected through various means, including, but not limited to, literature search, internet search, sequencing of genomic DNA, sequencing of an HLA library, and sequencing trios samples. Within the LD database, each association case is one row of alleles of different genes. For example, one row in the database is “A*01:01:01˜B*08:01:01˜C*07:01:01−269.101813335”, which suggests that the five alleles are associated with log(p-value)−−269.101813335. The lower the log(p-value), the stronger the association is. The LD database is then used to guide user to estimate the likelihood that a genotype of a particular sample is valid. To do so, other loci of the same sample must be genotyped or are known first. In some embodiments, the method involves trying to pair the target allele with known allele of different loci by the checking of the p-value of association of paired alleles. If there is no p-value or the p-value does not pass a specific cutoff, we will state that there is no statically significant association between paired alleles. By doing so, we can eliminate or at least lower the chance of mistyping samples.

In some embodiments, P-values are generated by computing a Chi-square value from real experiential data obtained in a population study.

In other embodiments, P-values are generated empirically.

TABLE 1
Examplary LD Association Table for Apecific
HLA Alleles

| Allele arrangement | −log(p-value) |
|---|---|
| A*01:01:01~B*08:01:01~C*07:01:01 | −269.101813335 |
| A*29:02:01~B*44:03:01~C*16:01:01 | −116.823554089 |
| A*03:01:01~B*07:02:01~C*07:02:01 | −90.0274318921 |
| A*33:01:01~B*14:02:01~C*08:02:01 | −69.9115076279 |
| A*02:01:01~B*44:02:01~C*05:01:01 | −58.2837914652 |
| A*26:01:01~B*38:01:01~C*12:03:01 | −57.6542687582 |
| A*25:01:01~B*18:01:01~C*12:03:01 | −55.506515017 |
| A*30:01:01~B*13:02:01~C*06:02:01 | −52.7838548781 |
| A*23:01:01~B*44:03:01~C*04:01:01 | −40.904853197 |
| A*11:01:01~B*35:01:01~C*04:01:01 | −38.6605427014 |

A first aspect of the present disclosure relates to a method for determining the association of Human Leukocyte Antigen (HLA) alleles at adjacent loci in genomic DNA from a biological sample obtained from a human subject. The method comprises the steps of amplifying a segment of genomic DNA from the sample by long-range PCR reaction; sequencing the amplified DNA; determining the association frequency of the genotype of an allele of a first locus with the genotype of an allele of at least one adjacent locus by reference to a database of loci associations; and reporting an association score of said allele in a first locus with the genotype of the allele in at least one adjacent locus, thereby determining the association of Human Leukocyte Antigen (HLA) alleles at adjacent loci in genomic DNA.

The association score is used to determine the correctness of an assignment of an allele by enumerating all possible combinations and choosing the combination with the lowest P-value score, i.e., the strongest association. A determination is then made as to whether the pairs from that lowest score can be found in the database. If the pairs are not found, they are flagged. Flagging is an indication that may be a warning that the sample was mis-typed, or that further study is required, as it may represent a previously unknown linkage pair. Accordingly, the association score is used to choose/predict the haplotype. After the haplotype has been chosen/predicted, the presence or absence of that haplotype in the database is determined.

In some embodiments, the association is determined between three or more loci. In some further embodiments, the two or more loci are in linkage disequilibrium with said first locus.

In other embodiments, the association is determined between four or more loci. In some further embodiments, the three or more loci are in linkage disequilibrium with the first locus.

In still other embodiments, the association is determined between five or more loci. In some further embodiments, the four or more loci are in linkage disequilibrium with the first locus.

In yet other embodiments, the association is determined between 11 loci. In some further embodiments, at least two of said eleven loci are in linkage disequilibrium.

In some embodiments, the first allele is an allele of an HLA locus selected from the group consisting of HLA-A, HLA-B, HLA-C, HLA-DRB1, HLA-DRB3, HLA-DRB4, HLA-DRB5, HLA-DQB1, HLA-DQA1, HLA-DPB1 and HLA-DPA1.

In some embodiments, the database of associations comprises associations of HLA loci for at least 2 loci selected from: HLA-A, HLA-B, HLA-C, HLA-DRB1, HLA-DRB3, HLA-DRB4, HLA-DRB5, HLA-DQB1, HLA-DQA1, HLA-DPB1 and HLA-DPA1.

In some embodiments, the at least one adjacent locus is in linkage disequilibrium with the first locus.

In some embodiments, the method further comprises comparing the association score to a database of association scores associated with a disease or disorder. In some further embodiments, the disease is an autoimmune disease. In some further embodiments, the autoimmune disease is selected from the group consisting of alopecia areata, autoimmune hemolytic anemia, autoimmune hepatitis, dermatomyositis, insulin dependent diabetes mellitus, autoimmune juvenile idiopathic arthritis, glomerulonephritis, Graves' disease, Guillain-Barré syndrome, idiopathic thrombocytopenic purpura, myasthenia gravis, autoimmune myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjogren's syndrome, systemic lupus erythematosus, some forms of thyroiditis, autoimmune uveitis, vitiligo, and Wegener's granulomatosis.

In other embodiments, the method further comprises comparing the association score to an association score obtained for a different human subject for assessing tissue compatibility.

In still other embodiments, the method further comprises determining the association of specific linked HLA alleles with the susceptibility to cancer. Exemplary cancers include: carcinoma, melanoma, sarcoma, lymphoma, leukemia, germ cell tumor, blastoma, and specific varieties thereof.

In yet other embodiments, the method further comprises determining the association of specific linked HLA alleles with responsiveness to therapeutic agents including, but not limited to, chemotherapeutic agents, immunotherapeutic agents, antibiotics, and anti-inflammatory agents.

In some embodiments, the segment of genomic DNA comprises at least one exon and one intron. In some embodiments, the segment of genomic DNA comprises at least two exons and one intron. In some embodiments, the segment of genomic DNA comprises at least two exons and two introns. In some embodiments, the segment of genomic DNA comprises at least three exons and two introns. In some embodiments, the segment of genomic DNA comprises at least three exons and three introns. In some embodiments, the segment of genomic DNA comprises at least four exons and three introns. In some embodiments, the segment of genomic DNA comprises at least four exons and four introns.

In some embodiments, the segment of genomic DNA comprises at least five exons and four introns. In some embodiments, the segment of genomic DNA comprises at least five exons and five introns. In some embodiments, the segment of genomic DNA comprises at least six exons and five introns. In some embodiments, the segment of genomic DNA comprises at least six exons and six introns. In some embodiments, the segment of genomic DNA comprises at least seven exons and six introns. In some embodiments, the segment of genomic DNA comprises at least seven exons and seven introns. In some embodiments, the segment of genomic DNA comprises at least eight exons and seven introns

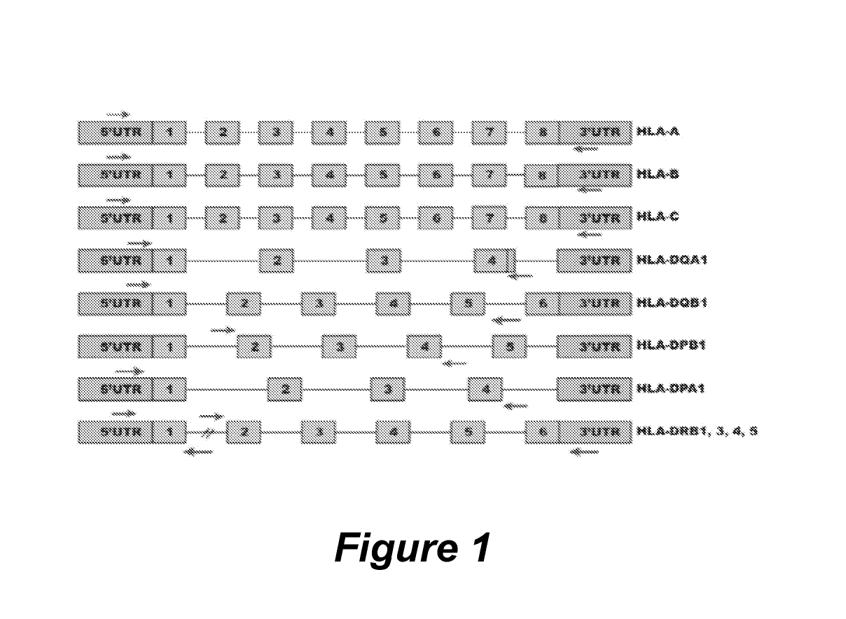

In some embodiments, the segment of genomic DNA comprises all of the exons of HLA-A. In some embodiments, the segment of genomic DNA comprises all of the exons of HLA-B. In some embodiments, the segment of genomic DNA comprises all of the exons of HLA-C. In some embodiments, the segment of genomic DNA comprises all of at exons 1, 2 and 3 and at least 75% of exon 4 of HLA-DQA1. In some embodiments, the segment of genomic DNA comprises all of at exons 1-5 of HLA-DQB1. In some embodiments, the segment of genomic DNA comprises all of at exons 2-4 of HLA-DPB1. In some embodiments, the segment of genomic DNA comprises all of at exons 1-4 of HLA-DPA1. In some embodiments, the segment of genomic DNA comprises all of at exons 2-6 of HLA-DRB1, HLA-DRB3, HLA-DRB4, or HLA-DRB5. Some exemplary schemes for long-range PCR amplification of HLA genes from human genomic DNA are shown in FIG. 1.

In other embodiments, the method further comprises amplifying at least one additional segment of genomic DNA from the sample by long-range PCR reaction; sequencing the amplified DNA of the at least one additional segment of genomic DNA; determining the association frequency of the genotype of an allele of a first locus with the genotype of an allele of at least one adjacent locus in the at least one additional segment of genomic DNA by reference to a database of loci associations; and reporting an association score of said allele in a first locus with the genotype of the allele in at least one adjacent locus, hereby determining an association of Human Leukocyte Antigen (HLA) alleles at adjacent loci in genomic DNA in the at least one additional segment of genomic DNA.

In still other embodiments, the method further comprises amplifying at least two, three, four, five, six or seven additional segments of genomic DNA from the sample by long-range PCR reaction; sequencing the amplified DNA of the at least two, three, four, five, six or seven additional segments of genomic DNA; determining the association frequency of the genotype of an allele of a first locus with the genotype of an allele of at least one adjacent locus in the at least two, three, four, five, six or seven additional segments of genomic DNA by reference to a database of loci associations; and reporting an association score of said allele in a first locus with the genotype of the allele in at least one adjacent locus, thereby determining an association of Human Leukocyte Antigen (HLA) alleles at adjacent loci in genomic DNA in the at least two, three, four, five, six or seven additional segments of genomic DNA.

A second aspect of the present disclosure relates to a database matrix for use in the analysis of association score of an allele to be assigned to a first locus and at least one additional locus. The database matrix comprises a field corresponding to an allele to be genotyped; at least one other field corresponding to another allele at a different locus; at least another field corresponding to the probability of the allele to be genotyped to a locus and the at least one other field as expressed as a probability.

A third aspect of the present disclosure relates to a method of validating correctness of an assignment of an allele variant to a genetic human leucocyte antigen (HLA) locus. The method comprises acquiring genotype information representing an assignment of an allele variant to a genetic human leucocyte antigen (HLA) locus; determining a score representing an association of the allele variant with at least one other allele variant of at least one adjacent genetic locus; using the determined score to generate the indication indicating correctness of the assignment of the allele variant to the genetic HLA locus; and reporting the indication.

In some embodiments, the method further comprises processing a biological sample.

In other embodiments, the method further comprises generating the genotype information.

A fourth aspect of the present disclosure relates to a method of operating a computing device comprising at least one processor, the method comprising executing the at least one processor to: acquire genotype information representing an assignment of an allele variant to a genetic human leukocyte antigen (HLA) locus; and when it is determined that a score representing an association of the allele variant with at least one other allele variant of at least one adjacent genetic locus exists: using the determined score to generate an indication indicating the likelihood of the correctness of the assignment of the allele variant to the genetic HLA locus; and reporting the indication.

In some embodiments, the at least one adjacent genetic locus comprises at least two adjacent genetic loci. In some further embodiments, the at least two adjacent genetic loci are in linkage disequilibrium database with the genetic HLA locus.

In other embodiments, the at least one adjacent genetic locus comprises three or more adjacent genetic loci. In some further embodiments, the at least three adjacent genetic loci are in linkage disequilibrium database with the genetic HLA locus.

In still other embodiments, the at least one adjacent genetic locus comprises four or more adjacent genetic loci. In some further embodiments, wherein the at least four adjacent genetic loci are in linkage disequilibrium database with the genetic HLA locus.

In yet other embodiments, the at least one adjacent genetic locus comprises five or more adjacent genetic loci. In some further embodiments, the at least five adjacent genetic loci are in linkage disequilibrium database with the genetic HLA locus.

In even other embodiments, the at least one adjacent genetic locus is an HLA locus.

In other embodiments, the allele variant is an allele of an HLA locus selected from: HLA-A, HLA-B, HLA-C, HLA-DRB1, HLA-DRB3, HLA-DRB4, HLA-DRB5, HLA-DQB1, HLA-DQA1, HLA-DPB1 and HLA-DPA1, where HLA-DRB3, HLA-DRB4 and HLA-DRB5 together are considered one locus.

In other embodiments, the method further comprises executing the at least one processor to determine whether the score exists by computing with information, by the at least one processor, a linkage disequilibrium database. In some further embodiments, the linkage disequilibrium database comprises associations of at least two HLA loci with one another, the at least two HLA loci being selected from: HLA-A, HLA-B, HLA-C, HLA-DRB1, HLA-DRB3, HLA-DRB4, HLA-DRB5, HLA-DQB1, HLA-DQA1, HLA-DPB1 and HLA-DPA1, where HLA-DRB3, HLA-DRB4 and HLA-DRB5 together are considered one locus. In other further embodiments, the method further comprises, when it is determined that the linkage disequilibrium database does not include the score, generating, by the at least one processor, a second indication indicating that the linkage disequilibrium database does not include the score. In still other further embodiments, the method further comprises, when it is determined that the linkage disequilibrium database does not include the score, flagging, by the at least one processor, at least one of the allele variant and the at least one other allele variant of the at least one adjacent genetic locus. In yet other further embodiments, the method further comprises, when it is determined that the linkage disequilibrium database does not include the score, flagging, by the at least one processor, at least one of the allele variant and the at least one other allele variant of the at least one adjacent genetic locus.

In some embodiments, the indication comprises a numerical value.

In other embodiments, the at least one adjacent genetic locus is in linkage disequilibrium database with the genetic HLA locus.

In some embodiments, reporting the indication comprises displaying a representation of the indication on a display device communicatively coupled with the computing device. In some further embodiments, the representation of the indication is displayed on the display device in a graphical format.

In some embodiments, the method further comprises acquiring second genotype information representing an assignment of the at least one other allele variant to the at least one adjacent genetic locus.

In other embodiments, the method further comprises acquiring the genotype information electronically via a network.

In some embodiments, the genotype information is generated using a genotyping technique that is different from a method of generating a genotype for an allele purely based on LD information.

In some embodiments, the method further comprises using the indication indicating correctness of the assignment of the allele variant to the genetic HLA locus to assess one or more of the following applications selected from the group consisting of: HLA typing, transplant capability, donor-recipient compatibility and diagnosis of graft versus host disease.

A fifth aspect of the present disclosure relates to a computing system comprising: at least one processor configured to assign at least one partial haplotype to a genetic locus by performing the method according to any one of the first through fourth aspects and their embodiments.

A sixth aspect of the present disclosure relates to a system for validating correctness of a genotype of a sample obtained from a subject, the system comprising: at least one processor; a memory communicatively coupled to the processor, the memory having stored thereon computer executable instructions that, when executed by the at least processor, perform a method comprising: acquiring genotype information representing an assignment of an allele variant to a genetic human leukocyte antigen (HLA) locus; determining a score representing an association of the allele variant with at least one other allele variant of at least one adjacent genetic locus; using the determined score to generate an indication indicating correctness of the assignment of the allele variant to the genetic HLA locus; and reporting the indication.

In some embodiments, the at least one adjacent genetic locus comprises at least two adjacent genetic loci.

In other embodiments, the at least one adjacent genetic locus is an HLA locus.

In some embodiments, the allele variant is an allele of an HLA locus selected from: HLA-A, HLA-B, HLA-C, HLA-DRB1, HLA-DRB3, HLA-DRB4, HLA-DRB5, HLA-DQB1, HLA-DQA1, HLA-DPB1 and HLA-DPA1, where HLA-DRB3, HLA-DRB4 and HLA-DRB5 together are considered one locus.

In some embodiments, determining the score comprises accessing a linkage disequilibrium database. In further some embodiments, the the linkage disequilibrium database comprises associations of at least two HLA loci with one another, the at least two HLA loci being selected from: HLA-A, HLA-B, HLA-C, HLA-DRB1, HLA-DRB3, HLA-DRB4, HLA-DRB5, HLA-DQB1, HLA-DQA1, HLA-DPB1 and HLA-DPA1, where HLA-DRB3, HLA-DRB4 and HLA-DRB5 together are considered one locus.

In some embodiments, the system further comprises acquiring second genotype information representing an assignment of the at least one other allele variant to the at least one adjacent genetic locus.

In other embodiments, the system further comprises acquiring the genotype information electronically via a network.

In some embodiments, the genotype information is generated using a genotyping technique that is different from a method of generating a genotype for an allele purely based on LD information.

In other embodiments, reporting the indication comprises displaying a representation of the indication on a display device communicatively coupled with the at least one processor. In some further embodiments, the representation of the indication is displayed on the display device in a graphical format.

A seventh aspect of the present disclosure relates to at least one non-transitory computer storage device storing computer-executable instructions that, when executed by at least one processor, cause the at least one processor to perform a method comprising: acquiring a genotype information representing an assignment of an allele variant to a genetic human leucocyte antigen (HLA) locus; acquiring a score representing an association of the allele variant with at least one other allele variant of at least one adjacent genetic locus; using the acquired score to generate an indication indicating correctness of the assignment of the allele variant to the genetic HLA locus; and reporting the indication.

In some embodiments, the at least one adjacent genetic locus comprises at least two adjacent genetic loci.

In some embodiments, the at least one adjacent genetic locus is an HLA locus.

In some embodiments, the allele variant is an allele of an HLA locus selected from: HLA-A, HLA-B, HLA-C, HLA-DRB1, HLA-DRB3, HLA-DRB4, HLA-DRB5, HLA-DQB1, HLA-DQA1, HLA-DPB1 and HLA-DPA1, where HLA-DRB3, HLA-DRB4 and HLA-DRB5 together are considered one locus.

In some embodiments, determining the score comprises accessing a linkage disequilibrium database. In some further embodiments, the linkage disequilibrium database comprises associations of at least two HLA loci with one another, the at least two HLA loci being selected from: HLA-A, HLA-B, HLA-C, HLA-DRB1, HLA-DRB3, HLA-DRB4, HLA-DRB5, HLA-DQB1, HLA-DQA1, HLA-DPB1 and HLA-DPA1, where HLA-DRB3, HLA-DRB4 and HLA-DRB5 together are considered one locus.

In some embodiments, the method further comprises acquiring second genotype information representing an assignment of the at least one other allele variant to the at least one adjacent genetic locus.

In some embodiments, the method further comprises acquiring the genotype information electronically via a network.

In some embodiments, the genotype information is generated using a genotyping technique comprising the steps of: acquiring genotype information representing an assignment of an allele variant to a genetic human leukocyte antigen (HLA) locus; acquiring second genotype information representing an assignment of the at least one other allele variant to the at least one adjacent genetic locus; determining a score representing an association of the allele variant with at least one other allele variant of at least one adjacent genetic locus; using the determined score to generate an indication indicating correctness of the assignment of the allele variant to the genetic HLA locus; and reporting the indication.

An eighth aspect of the present disclosure relates to a method of operating a computing device comprising at least one processor, the method comprising executing the at least one processor to: acquire genotype information representing an assignment of an allele variant to a genetic human leucocyte antigen (HLA) locus; accessing a computer readable storage device storing linkage disequilibrium information to determine whether the linkage disequilibrium information includes a score representing an association of the allele variant with at least one other allele variant of at least one adjacent genetic locus; when it is determined that the linkage disequilibrium information includes the score: using the determined score to generate an indication indicating correctness of the assignment of the allele variant to the genetic HLA locus; and reporting the indication.

In some embodiments, the method further comprises, when it is determined that the linkage disequilibrium information does not include the score, generating an indication indicating that the linkage disequilibrium information does not include the score. In some further embodiments, the method further comprises, when it is determined that the linkage disequilibrium information does not include the score, flagging at least one of the allele variant and the at least one other allele variant of the at least one adjacent genetic locus.

In some embodiments, reporting the indication comprises displaying a representation of the indication on a display device communicatively coupled with the computing device.

In other embodiments, the representation of the indication is displayed on the display device in a graphical format.

A ninth aspect of the present disclosure relates to a method of assigning an allele to a genetic locus comprising: amplifying coding and non-coding DNA from a genetic locus from a sample of genomic DNA to produce an amplicon; sequencing the amplicon; identifying at least a first allele variant and a second allele variant of the genetic locus from the amplicon; determining a score representing an association of the first allele variant with at least one other allele variant of at least one adjacent genetic locus; and using the determined score to generate an indication indicating correctness of the assignment of the allele variant to the genetic HLA locus.

In some embodiments, the coding and non-coding DNA are from an exon and an adjacent intron.

In other embodiments, the sequencing is done by a next generation sequencing method.

In some embodiments, the coding DNA comprises at least two exons.

In other embodiments, the coding DNA comprises at least three exons.

In still other embodiments, the coding DNA comprises at least four exons.

In some embodiments, the non-coding DNA comprises at least one intron.

In other embodiments, the non-coding DNA comprises at least two introns.

In still other embodiments, the non-coding DNA comprises at least three introns.

In yet other embodiments, the non-coding DNA comprises at least four introns.

In some embodiments, the at least one adjacent genetic locus is in linkage disequilibrium database with the genetic HLA locus.

In some embodiments, the at least one adjacent genetic locus comprises at least two adjacent genetic loci. In some further embodiments, the at least two adjacent genetic loci are in linkage disequilibrium database with the genetic HLA locus.

In other embodiments, the at least one adjacent genetic locus comprises three or more adjacent genetic loci. In some further embodiments, the at least three adjacent genetic loci are in linkage disequilibrium database with the genetic HLA locus.

In still other embodiments, the at least one adjacent genetic locus comprises four or more adjacent genetic loci. In some further embodiments, the at least four adjacent genetic loci are in linkage disequilibrium database with the genetic HLA locus.

In yet other embodiments, the at least one adjacent genetic locus comprises five or more adjacent genetic loci. In some further embodiments, the at least five adjacent genetic loci are in linkage disequilibrium database with the genetic HLA locus.

In some embodiments, the at least one adjacent genetic locus is an HLA locus.

In some embodiments, the allele variant is an allele of an HLA locus selected from: HLA-A, HLA-B, HLA-C, HLA-DRB1, HLA-DRB3, HLA-DRB4, HLA-DRB5, HLA-DQB1, HLA-DQA1, HLA-DPB1 and HLA-DPA1, where HLA-DRB3, HLA-DRB4 and HLA-DRB5 together are considered one locus.

In some embodiments, determining the score comprises accessing a linkage disequilibrium database. In some further embodiments, the linkage disequilibrium database comprises associations of at least two HLA loci with one another, the at least two HLA loci being selected from: HLA-A, HLA-B, HLA-C, HLA-DRB1, HLA-DRB3, HLA-DRB4, HLA-DRB5, HLA-DQB1, HLA-DQA1, HLA-DPB1 and HLA-DPA1, where HLA-DRB3, HLA-DRB4 and HLA-DRB5 together are considered one locus.

High resolution HLA typing is a powerful method to characterize HLA allele and haplotype diversity in population studies. Whole gene coverage provided extensive polymorphic sites that define the physical linkage between exons and helps to resolve trans, or combination ambiguities in phasing. HLA typing to single nucleotide resolution allowed detection of previously unreported variants, including coding sequence changes

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present disclosure. The disclosure is intended to cover the components and steps in any sequence which is effective to meet the objectives there intended, unless the context specifically indicates the contrary.

What is claimed is:

1. A method for determining the association of Human Leukocyte Antigen (HLA) alleles at adjacent loci in genomic DNA from a biological sample obtained from a human subject, comprising:
a. amplifying a segment of genomic DNA from the sample by long-range PCR reaction;
b. sequencing the amplified DNA;
c. determining the association frequency of the genotype of an allele of a first locus with the genotype of an allele of at least one adjacent locus by reference to a database of loci associations; and
d. reporting an association score of said allele in a first locus with the genotype of the allele in at least one adjacent locus, thereby determining an association of Human Leukocyte Antigen (HLA) alleles at adjacent loci in genomic DNA.

2. The method of claim 1, wherein said association is determined between three or more loci.

3. The method of claim 1, wherein said association is determined between four or more loci.

4. The method of claim 1, wherein said association is determined between five or more loci.

5. The method of claim 1, wherein said association is determined between 11 loci.

6. The method of claim 1, wherein said first allele is an allele of an HLA locus selected from the group consisting of HLA-A, HLA-B, HLA-C, HLA-DRB1, HLA-DRB3, HLA-DRB4, HLA-DRB5, HLA-DQB1, HLA-DQA1, HLA-DPB1 and HLA-DPA1.

7. The method of claim 1, wherein said database of associations comprises associations of HLA loci for at least 2 loci selected from: HLA-A, HLA-B, HLA-C, HLA-DRB1, HLA-DRB3, HLA-DRB4, HLA-DRB5, HLA-DQB1, HLA-DQA1, HLA-DPB1 and HLA-DPA 1.

8. The method of claim 1, wherein the at least one adjacent locus is in linkage disequilibrium with the first locus.

9. The method of claim 2, wherein at least one of said two or more loci are in linkage disequilibrium with said first locus.

10. The method of claim 3, wherein at least one of said three or more loci are in linkage disequilibrium with the first locus.

11. The method of claim 4, wherein at least one of said four or more loci are in linkage disequilibrium with the first locus.

12. The method of claim 5, wherein at least two of said eleven loci are in linkage disequilibrium.

13. The method of claim 1, further comprising comparing the association score to a database of association scores associated with a disease.

14. The method of claim 13, wherein the disease is an autoimmune disease.

15. The method of claim 1, further comprising comparing the association score to an association score obtained for a different human subject for assessing tissue compatibility.

16. A database matrix for use in the analysis of association score of an allele to be assigned to a first locus and at least one additional locus, said database matrix comprising:
a. a field corresponding to an allele to be genotyped;
b. at least one other field corresponding to another allele at a different locus;
c. at least another field corresponding to the probability of the allele to be genotyped to a locus and the at least one other field as expressed as a probability.

17. A method of validating correctness of an assignment of an allele variant to a genetic human leucocyte antigen (HLA) locus, the method comprising:
acquiring genotype information representing an assignment of an allele variant to a genetic human leucocyte antigen (HLA) locus;
determining a score representing an association of the allele variant with at least one other allele variant of at least one adjacent genetic locus;
generating an indication based upon the determined score indicating correctness of the assignment of the allele variant to the genetic HLA locus; and
reporting the indication.

18. The method of claim 17, further comprising processing a biological sample.

19. The method of claim 17, further comprising generating the genotype information.

20. A method of operating a computing device comprising at least one processor, the method comprising:
executing the at least one processor to acquire genotype information representing an assignment of an allele variant to a genetic human leukocyte antigen (HLA) locus; and
when it is determined that a score representing an association of the allele variant with at least one other allele variant of at least one adjacent genetic locus exists:
generating an indication upon the determined score indicating the likelihood of the correctness of the assignment of the allele variant to the genetic HLA locus; and
reporting the indication.

21. The method of claim 20, wherein the at least one adjacent genetic locus comprises at least two adjacent genetic loci.

22. The method of claim 20, wherein the at least one adjacent genetic locus comprises three or more adjacent genetic loci.

23. The method of claim 20, wherein the at least one adjacent genetic locus comprises four or more adjacent genetic loci.

24. The method of claim 20, wherein the at least one adjacent genetic locus comprises five or more adjacent genetic loci.

25. The method of claim 20, wherein the at least one adjacent genetic locus is an HLA locus.

26. The method of claim 20, wherein the allele variant is an allele of an HLA locus selected from: HLA-A, HLA-B, HLA-C, HLA-DRB1, HLA-DRB3, HLA-DRB4, HLA-DRB5, HLA-DQB1, HLA-DQA1, HLA-DPB1 and HLA-DPA1, where HLA-DRB3, HLA-DRB4 and HLA-DRB5 together are considered one locus.

27. The method of claim 20, further comprising executing the at least one processor to determine whether the score exists by computing with information, by the at least one processor, in a linkage disequilibrium database.

28. The method of claim 27, wherein the linkage disequilibrium database comprises associations of at least two HLA loci with one another, the at least two HLA loci being selected from: HLA-A, HLA-B, HLA-C, HLA-DRB1, HLA-DRB3, HLA-DRB4, HLA-DRB5, HLA-DQB1, HLA-DQA1, HLA-DPB1 and HLA-DPA1, where HLA-DRB3, HLA-DRB4 and HLA-DRB5 together are considered one locus.

29. The method of claim 27, further comprising, when it is determined that the linkage disequilibrium database does not include the score, generating, by the at least one processor, a second indication indicating that the linkage disequilibrium database does not include the score.

30. The method of claim 27, further comprising, when it is determined that the linkage disequilibrium database does not include the score, flagging, by the at least one processor, at least one of the allele variant and the at least one other allele variant of the at least one adjacent genetic locus.

31. The method of claim 27, further comprising, when it is determined that the linkage disequilibrium database does not include the score, flagging, by the at least one processor, at least one of the allele variant and the at least one other allele variant of the at least one adjacent genetic locus.

32. The method of claim 20, wherein the indication comprises a numerical value.

33. The method of claim 20, wherein the at least one adjacent genetic locus is in linkage disequilibrium with the genetic HLA locus.

34. The method of claim 21, wherein the at least two adjacent genetic loci are in linkage disequilibrium database with the genetic HLA locus.

35. The method of claim 22, wherein the at least three adjacent genetic loci are in linkage disequilibrium database with the genetic HLA locus.

36. The method of claim 23, wherein the at least three adjacent genetic loci are in linkage disequilibrium database with the genetic HLA locus.

37. The method of claim 24, wherein the at least three adjacent genetic loci are in linkage disequilibrium database with the genetic HLA locus.

38. The method of claim 20, wherein reporting the indication comprises displaying a representation of the indication on a display device communicatively coupled with the computing device.

39. The method of claim 38, wherein the representation of the indication is displayed on the display device in a graphical format.

40. The method of claim 20, further comprising acquiring second genotype information representing an assignment of the at least one other allele variant to the at least one adjacent genetic locus.

41. The method of claim 20, comprising acquiring the genotype information electronically via a network.

42. The method of claim 20, wherein the genotype information is generated using a genotyping technique that is different from the method of claim 1.

43. The method of claim 20, further comprising using the indication indicating correctness of the assignment of the allele variant to the genetic HLA locus to assess one or more of the following applications selected from the group consisting of: HLA typing, transplant capability, donor-recipient compatibility and diagnosis of graft versus host disease.

44. A computing system comprising:
at least one processor configured to assign at least one partial haplotype to a genetic locus by performing the method according to any one of claims 20-43.

45. A system for validating correctness of a genotype of a sample obtained from a subject, the system comprising:
at least one processor;
a memory communicatively coupled to the processor, the memory having stored thereon computer executable instructions that, when executed by the at least processor, perform a method comprising:
acquiring genotype information representing an assignment of an allele variant to a genetic human leukocyte antigen (HLA) locus;
determining a score representing an association of the allele variant with at least one other allele variant of at least one adjacent genetic locus;
generating an indication based upon the determined score indicating correctness of the assignment of the allele variant to the genetic HLA locus; and
reporting the indication.

46. The system of claim 45, wherein the at least one adjacent genetic locus comprises at least two adjacent genetic loci.

47. The system of claim 45, wherein the at least one adjacent genetic locus is an HLA locus.

48. The system of claim 45, wherein the allele variant is an allele of an HLA locus selected from: HLA-A, HLA-B, HLA-C, HLA-DRB1, HLA-DRB3, HLA-DRB4, HLA-DRB5, HLA-DQB1, HLA-DQA1, HLA-DPB1 and HLA-DPA1, where HLA-DRB3, HLA-DRB4 and HLA-DRB5 together are considered one locus.

49. The system of claim 45, wherein determining the score comprises accessing a linkage disequilibrium database.

50. The system of claim 49, wherein the linkage disequilibrium database comprises associations of at least two HLA loci with one another, the at least two HLA loci being selected from: HLA-A, HLA-B, HLA-C, HLA-DRB1, HLA-DRB3, HLA-DRB4, HLA-DRB5, HLA-DQB1, HLA-DQA1, HLA-DPB1 and HLA-DPA1, where HLA-DRB3, HLA-DRB4 and HLA-DRB5 together are considered one locus.

51. The system of claim 45, wherein the method further comprises acquiring second genotype information representing an assignment of the at least one other allele variant to the at least one adjacent genetic locus.

52. The system of claim 45, wherein the method further comprises acquiring the genotype information electronically via a network.

53. The system of claim 45, wherein the genotype information is generated using a genotyping technique that is different from the method of claim 1.

54. The system of claim 45, wherein reporting the indication comprises displaying a representation of the indication on a display device communicatively coupled with the at least one processor.

55. The system of claim 45, wherein the representation of the indication is displayed on the display device in a graphical format.

56. At least one non-transitory computer storage device storing computer-executable instructions that, when executed by at least one processor, cause the at least one processor to perform a method comprising:
acquiring a genotype information representing an assignment of an allele variant to a genetic human leucocyte antigen (HLA) locus;
acquiring a score representing an association of the allele variant with at least one other allele variant of at least one adjacent genetic locus;
generating an indication based upon the acquired score indicating correctness of the assignment of the allele variant to the genetic HLA locus; and
reporting the indication.

57. The at least one non-transitory computer storage device of claim 56, wherein the at least one adjacent genetic locus comprises at least two adjacent genetic loci.

58. The at least one non-transitory computer storage device of claim 56, wherein the at least one adjacent genetic locus is an HLA locus.

59. The at least one non-transitory computer storage device of claim 56, wherein the allele variant is an allele of an HLA locus selected from: HLA-A, HLA-B, HLA-C, HLA-DRB1, HLA-DRB3, HLA-DRB4, HLA-DRB5, HLA-DQB1, HLA-DQA1, HLA-DPB1 and HLA-DPA1, where HLA-DRB3, HLA-DRB4 and HLA-DRB5 together are considered one locus.

60. The at least one non-transitory computer storage device of claim 56, wherein determining the score comprises accessing a linkage disequilibrium database.

61. The at least one non-transitory computer storage device of claim 60, wherein the linkage disequilibrium database comprises associations of at least two HLA loci with one another, the at least two HLA loci being selected from: HLA-A, HLA-B, HLA-C, HLA-DRB1, HLA-DRB3, HLA-DRB4, HLA-DRB5, HLA-DQB1, HLA-DQA1, HLA-DPB1 and HLA-DPA1, where HLA-DRB3, HLA-DRB4 and HLA-DRB5 together are considered one locus.

62. The at least one non-transitory computer storage device of claim 56, wherein the method further comprises acquiring second genotype information representing an assignment of the at least one other allele variant to the at least one adjacent genetic locus.

63. The at least one non-transitory computer storage device of claim 56, wherein the method further comprises acquiring the genotype information electronically via a network.

64. The at least one non-transitory computer storage device of claim 56, wherein the genotype information is generated using a genotyping technique comprising the steps of: acquiring genotype information representing an assignment of an allele variant to a genetic human leukocyte antigen (HLA) locus; acquiring second genotype information representing an assignment of the at least one other allele variant to the at least one adjacent genetic locus; determining a score representing an association of the allele variant with at least one other allele variant of at least one adjacent genetic locus; using the determined score to generate an indication indicating correctness of the assignment of the allele variant to the genetic HLA locus; and reporting the indication.

65. A method of operating a computing device comprising at least one processor, the method comprising executing the at least one processor to:
acquire genotype information representing an assignment of an allele variant to a genetic human leucocyte antigen (HLA) locus;
accessing a computer readable storage device storing linkage disequilibrium information to determine whether the linkage disequilibrium information includes a score representing an association of the allele variant with at least one other allele variant of at least one adjacent genetic locus;
when it is determined that the linkage disequilibrium information includes the score;
generating an indication based upon the determined score indicating correctness of the assignment of the allele variant to the genetic HLA locus; and
reporting the indication.

66. The method of claim 65, further comprising, when it is determined that the linkage disequilibrium information does not include the score, generating an indication indicating that the linkage disequilibrium information does not include the score.

67. The method of claim 66, further comprising, when it is determined that the linkage disequilibrium information does not include the score, flagging at least one of the allele variant and the at least one other allele variant of the at least one adjacent genetic locus.

68. The method of claim 65, wherein reporting the indication comprises displaying a representation of the indication on a display device communicatively coupled with the computing device.

69. The method of claim 65, wherein the representation of the indication is displayed on the display device in a graphical format.

70. A method of assigning an allele to a genetic locus comprising:
amplifying coding and non-coding DNA from a genetic locus from a sample of genomic DNA to produce an amplicon
sequencing the amplicon;
identifying at least a first allele variant and a second allele variant of the genetic locus from the amplicon;
determining a score representing an association of the first allele variant with at least one other allele variant of at least one adjacent genetic locus; and
generating an indication based upon the determined score indicating correctness of the assignment of the allele variant to the genetic HLA locus.

71. The method of claim 70, wherein said coding and non-coding DNA are from an exon and an adjacent intron.

72. The method of claim 70, wherein the sequencing is done by a next generation sequencing method.

73. The method of claim 70, wherein said coding DNA comprises at least two exons.

74. The method of claim 70, wherein said coding DNA comprises at least two exons

75. The method of claim 70, wherein said coding DNA comprises at least two exons.

76. The method of claim 70, wherein said non-coding DNA comprises at least one intron.

77. The method of claim 70, wherein said non-coding DNA comprises at least two introns.

78. The method of claim 70, wherein said non-coding DNA comprises at least two introns.

79. The method of claim 70, wherein said non-coding DNA comprises at least three introns.

80. The method of claim 70, wherein the at least one adjacent genetic locus comprises at least two adjacent genetic loci.

81. The method of claim 70, wherein the at least one adjacent genetic locus comprises three or more adjacent genetic loci.

82. The method of claim 70, wherein the at least one adjacent genetic locus comprises four or more adjacent genetic loci.

83. The method of claim 70, wherein the at least one adjacent genetic locus comprises five or more adjacent genetic loci.

84. The method of claim 70, wherein the at least one adjacent genetic locus is an HLA locus.

85. The method of claim 70, wherein the allele variant is an allele of an HLA locus selected from: HLA-A, HLA-B, HLA-C, HLA-DRB1, HLA-DRB3, HLA-DRB4, HLA-DRB5, HLA-DQB1, HLA-DQA1, HLA-DPB1 and HLA-DPA1, where HLA-DRB3, HLA-DRB4 and HLA-DRB5 together are considered one locus.

86. The method of claim 70, wherein determining the score comprises accessing a linkage disequilibrium database.

87. The method of claim 86, wherein the linkage disequilibrium database comprises associations of at least two HLA loci with one another, the at least two HLA loci being selected from: HLA-A, HLA-B, HLA-C, HLA-DRB1, HLA-DRB3, HLA-DRB4, HLA-DRB5, HLA-DQB1, HLA-DQA1, HLA-DPB1 and HLA-DPA1, where HLA-DRB3, HLA-DRB4 and HLA-DRB5 together are considered one locus.

88. The method of any one of claims 70-86 wherein the at least one adjacent genetic locus is in linkage disequilibrium database with the genetic HLA locus.

89. The method of claim 80, wherein the at least two adjacent genetic loci are in linkage disequilibrium database with the genetic HLA locus.

90. The method of claim 81, wherein the at least three adjacent genetic loci are in linkage disequilibrium database with the genetic HLA locus.

91. The method of claim 82, wherein the at least four adjacent genetic loci are in linkage disequilibrium database with the genetic HLA locus.

92. The method of claim 83, wherein the at least five adjacent genetic loci are in linkage disequilibrium database with the genetic HLA locus.
